(19) United States
(12) Patent Application Publication
(10) Pub. No.: US 2021/0148933 A1
(43) Pub. Date: May 20, 2021

(54) METHODS AND ASSAYS FOR USE IN DIAGNOSIS OF LAMINITIS

(71) Applicant: PEPSIS LIMITED, Ledbury (GB)

(72) Inventors: Rosemary Waring, Ledbury (GB); John Hunter, Ledbury (GB)

(21) Appl. No.: 17/041,888

(22) PCT Filed: Mar. 26, 2019
(86) PCT No.: PCT/GB2019/050860

§ 371 Date: Sep. 25, 2020

(30) Foreign Application Priority Data
Mar. 26, 2018 (GB) 1804837.1

(51) Int. Cl. G01N 33/68

(52) U.S. Cl. CPC G01N 33/6893

(57) ABSTRACT

The invention concerns a method and assay for qualifying equine laminitis disease status in a subject by measuring specific biomarkers, comparing the level of said biomarker to a control level; and correlating the measurement with laminitis disease status, wherein a statistically relevant elevated level of said biomarker is present in said sample relative to said control level. The invention further relates to immuno-based methods and assays for detecting toxins relevant to laminitis and to the use of antibodies for assay and diagnostic purposes.

The present invention relates to assays and methods useful in the qualification and/or diagnosis of laminitis, frequently equine laminitis. The invention further relates to methods and assays for either detecting toxins relevant to laminitis and its disease pathway, or diagnosing laminitis from a sample. The invention also concerns compositions comprising agents which possess specific or particular anti-toxic capabilities in combination with enzyme rich malt extracts for use in the treatment of laminitis.

### BACKGROUND

Laminitis is a serious condition commonly associated with lameness and disability of horses and ponies. The condition presents as an inflammatory response in the sub-mural laminar structures of the hoof. The tissue of an inflicted horse typically evidences an inflammatory response in the digital laminar tissues and other anatomical structures within the hoof such as the inter-laminar bond between the dermal and epidermal laminae which supports the distal phalanx within the hoof. If the inter-laminar bonds are degraded the animal may become foundered and the pedal bone may move distally within the hoof. The physiological damage occurs very quickly rendering the animal irreversibly disabled. The condition is very painful and the horse may be euthanased since therapeutic approaches are often unsatisfactory. Thus, an easy and efficient method of determination or detection of this pathology in equines is very desirable as it could prevent disease, death, disease progression and/or aid recovery. This is particularly true of thorough-bred horses where quick detection or prevention methods may protect a very valuable horse.

The mechanism causing laminitis is still not completely understood; the symptoms and effects of treatments have previously varied greatly amongst veterinarians. Thus, there is a distinct need for a greater understanding of the underlying causes of this condition and an effective method of diagnosis, prevention and/or treatment. Many therapeutic regimes have been described to treat laminitis but very few reports are able to support their technical theory or biological links with clear evidence.

Fructans, a grass sugar, has been examined as a potential source of carbohydrate which is considered to increase susceptibility to laminitis. It has been acknowledge for some time that an ‘excess’ ingestion of such sugar or carbohydrate may impact “metabolic syndrome” in horses and this may be linked to clinical signs of laminitis (Pollitt (2003) Hoof care and Lameness Journal 76: 20-22; Pollitt (2001) “Equine Laminitis”, Kingston, Rural Industries Research) and (Development Corporation and Johnson (2002) Vet. Clin. Equine 18: 271-293; 219-236).

In particular, authors in the field have commented that transient disruption of the ratios between chemicals, such as indole amines (serotonin and melatonin) and catecholamine (dopamine) have a part to play as the progressive degeneration of the pineal gland causes a change in relative ratios of these chemicals.

In some cases researchers have implicated oxidative stress (Laskowski L M et al 2016 Vet Immunol Immunopathol 171 66-72) and insulin dysregulation (de Laat M A et al 2016 Am J Physiol—Endocrinol Metab 310 (1) E61-72), where there is a resistance to the action of insulin coupled with high levels of the hormone. However, this does not explain the reported links with feeding on excess carbohydrate/new grass or gastrointestinal disorders (Onishi J C et al 2012 Vet Microbiol 159 (3-4) 354-63 and Harlow B E et al 2014 J Appl Microbiol 117 (2) 329-339).

Laminitis therefore appears to be a complex, multi-factorial problem and to date, there is still no concrete theory which has been evidenced or has resulted in a useful diagnostic method, tool or treatment in laminitis or related conditions. Thus, a method and/or assay useful in qualifying the disease or helping prevent increased risk of disease development remains highly desirable in the field of equine health.

It would be greatly beneficial, particularly in the field of horseracing, to accurately determine if such a horse would benefit from treatment, intervention or prevention. Furthermore, it would be very useful to present new treatment possibilities that have not been previously considered.

It is in view of these continuing needs, particularly as they present in the equine field, the present invention has been developed from further focussed research by the applicant.

### SUMMARY

The present invention concerns a method for qualifying equine laminitis disease status in a subject comprising: measuring at least 1 biomarker in a faecal sample from the subject, wherein said chemical biomarker is selected from:

Heptadecasphinganine (2S,3R)-2-aminoheptadecane-1,3-diol) “C17 Sphinganine”;

4-Hydroxyestrone sulfate AND/OR 1-O-alpha-D-Glucopyranosyl-D-mannitol AND/OR Melibiitol AND/OR (1(10)E,6a,9b)-9-(2-Methylpropanoyloxy)-1(10),4,11(13)-germacratrien-12,6-olide AND/OR Ubiquinone-2;

Citronellyl beta-sophoroside AND/OR 1alpha,11alpha,25-trihydroxycholecalciferol AND/OR 1alpha,11beta,25-trihydroxycholecalciferol AND/OR 1alpha,18,25-trihydroxycholecalciferol AND/OR 1alpha,2,25-trihydroxycholecalciferol AND/OR 1alpha,20,25-trihydroxycholecalciferol AND/OR 1alpha,22,25-trihydroxy-20-epicholecalciferol AND/OR 1alpha,23,25-trihydroxycholecalciferol AND/OR 1alpha,24,25-trihydroxycholecalciferol AND/OR 1alpha,25-dihydroxy-10,19-methano-23-oxacholecalciferol AND/OR 1alpha,25-dihydroxy-24a-homo-22-oxa-20-epicholecalciferol AND/OR 1alpha,25-dihydroxy-24a-homo-22-oxacholecalciferol AND/OR 1alpha,25,26-trihydroxycholecalciferol AND/OR 1alpha,26-trihydroxycholecalciferol AND/OR 22,24,25-trihydroxycholecalciferol AND/OR 23,24,25-trihydroxycholecalciferol AND/OR 23,25,26-trihydroxycholecalciferol AND/OR 24,25,26-trihydroxycholecalciferol AND/OR 6,19-epidioxy-1alpha-hydroxy-6,19-dihydrocholecalciferol AND/OR 6,19-epidioxy-25-hydroxy-6,19-dihydrocholecalciferol AND/OR 24-Hydroxycalcitriol AND/OR 3 beta,7 alpha-Dihydroxy-5-cholestenoate AND/OR 3beta,7alpha-dihydroxycholest-5-en-27-oic acid AND/OR 5beta-spirostan-1beta,3alpha-diol AND/OR 7alpha,12alpha,24-trihydroxycholest-4-en-3-one AND/OR 7alpha,12alpha,26-trihydroxycholest-4-en-3-one;

10beta-Hydroxy-6beta-isobutyrylfuranoeremophilane AND/OR 5′-carboxy-alpha-chromanol AND/OR QH2 AND/OR Testolate;

1alpha,19,25-trihydroxy-10,19-dihydrocholecalciferol AND/OR 1alpha,25-dihydroxy-2alpha-hydroxymethyl-19-nor-20-epicholecalciferol AND/OR 1alpha,25-dihydroxy-2alpha-hydroxymethyl-19-norcholecalciferolcholecalciferol AND/OR 1alpha,25-dihydroxy-2beta-hydroxymethyl-19-nor-20-epicholecalciferol AND/OR 1alpha,25-dihydroxy-2beta-hydroxymethyl-19-norcholecalciferol AND/OR 3a,7a-Dihydroxycoprostanic acid AND/OR 3a,7a,12a-Trihydroxy-5b-cholestan-26-al AND/OR 3alpha,7alpha-dihydroxy-5beta-cholestan-26-oic acid AND/OR 3alpha,7alpha,12alpha-trihydroxy-5beta-cholestan-26-al AND/OR 3alpha,7alpha,24-trihydroxy-5beta-cholestan-26-al AND/OR 7alpha,12alpha,24-trihydroxy-5beta-cholestan-3-one AND/OR 7alpha,25,27-trihydroxycholesterol;

comparing the level of said biomarker to a control level; and correlating the measurement with laminitis disease status, wherein a statistically relevant elevated level of said biomarker is present in said sample relative to said control level.

The inventors have concluded that at least 5 unique metabolite features showed a statistically significant higher concentration in laminitis horse faecal matter compared to control horses and as such one (or more) of such biomarkers is a powerful indication of laminitis pathogenesis.

The applicant believes that these results evidence a clear link between diagnoses of laminitis and chemical metabolite profiling difference in a horse's faecal matter as compared to a healthy animal.

However, in embodiments, the at least one biomarker is C17 Sphinganine. Of all the metabolites, C17 Sphinganine showed the largest statistically different change in concentration, where the value was 20 times the control value in the laminitic horses.

This chemical known as Heptadecasphinganine or (2S,3R)-2-aminoheptadecane-1,3-diol having the formula C17H37NO2 is therefore considered very significant.

It has been observed by the applicants that a particular known toxin, fumonisin or FB1, discussed in below, has a structure chemical similar to that of Sphinganine. Without being bound by theory the applicants suggest this toxin may act to inhibit the incorporation of C17 Sphinganine into sphingolipids (by inhibition of the enzyme ceramide synthase). As will be explained in further detail, where there is gut inflammation from excess carbohydrate intake and an increased permeability of the gut wall, the ingestion of FB1 means that the effect is exacerbated and the toxin rapidly penetrates the systemic circulation with devastating effect. This combination of events leads to a dramatically increased level of a specifically identifiable Sphinganine, as excess levels build up in vivo and has been recorded by the present applicants in their study. Sphinganine levels are therefore notably raised due to the ingestion of this toxin.

In a further aspect of the invention there is provided a method for qualifying equine laminitis disease status in a subject, wherein the method comprises identifying a combination of at least 2 different metabolic biomarkers in a faecal sample from the subject, wherein the at least 2 said chemical biomarkers are selected from:

Heptadecasphinganine (2S,3R)-2-aminoheptadecane-1,3-diol “C17 Sphinganine”; AND 4-Hydroxyestrone sulfate AND/OR 1-O-alpha-D-Glucopyranosyl-D-mannitol AND/OR Melibiitol AND/OR (1(10)E,6a,9b)-9-(2-Methylpropanoyloxy)-1(10),4,11(13)-germacratrien-12,6-olide AND/OR Ubiquinone-2;

Citronellyl beta-sophoroside AND/OR 1alpha,11alpha,25-trihydroxycholecalciferol AND/OR 1alpha,11beta,25-trihydroxycholecalciferol AND/OR 1alpha,18,25-trihydroxycholecalciferol AND/OR 1alpha,2,25-trihydroxycholecalciferol AND/OR 1alpha,20,25-trihydroxycholecalciferol AND/OR 1alpha,22,25-trihydroxy-20-epicholecalciferol AND/OR 1alpha,23,25-trihydroxycholecalciferol AND/OR 1alpha,24,25-trihydroxycholecalciferol AND/OR 1alpha,25-dihydroxy-10,19-methano-23-oxacholecalciferol AND/OR 1alpha,25-dihydroxy-24a-homo-22-oxa-20-epicholecalciferol AND/OR 1alpha,25-dihydroxy-24a-homo-22-oxacholecalciferol AND/OR 1alpha,25,26-trihydroxycholecalciferol AND/OR 1alpha,26-trihydroxycholecalciferol AND/OR 22,24,25-trihydroxycholecalciferol AND/OR 23,24,25-trihydroxycholecalciferol AND/OR 23,25,26-trihydroxycholecalciferol AND/OR 24,25,26-trihydroxycholecalciferol AND/OR 6,19-epidioxy-1alpha-hydroxy-6,19-dihydrocholecalciferol AND/OR 6,19-epidioxy-25-hydroxy-6,19-dihydrocholecalciferol AND/OR 24-Hydroxycalcitriol AND/OR 3 beta,7 alpha-Dihydroxy-5-cholestenoate AND/OR 3beta,7alpha-dihydroxycholest-5-en-27-oic acid AND/OR 5beta-spirostan-1beta,3alpha-diol AND/OR 7alpha,12alpha,24-trihydroxycholest-4-en-3-one AND/OR 7alpha,12alpha,26-trihydroxycholest-4-en-3-one;

10beta-Hydroxy-6beta-isobutyrylfuranoeremophilane AND/OR 5′-carboxy-alpha-chromanol AND/OR QH2 AND/OR Testolate;

1alpha,19,25-trihydroxy-10,19-dihydrocholecalciferol AND/OR 1alpha,25-dihydroxy-2alpha-hydroxymethyl-19-nor-20-epicholecalciferol AND/OR 1alpha,25-dihydroxy-2alpha-hydroxymethyl-19-norcholecalciferolcholecalciferol AND/OR 1alpha,25-dihydroxy-2beta-hydroxymethyl-19-nor-20-epicholecalciferol AND/OR 1alpha,25-dihydroxy-2beta-hydroxymethyl-19-norcholecalciferol AND/OR 3a,7a-Dihydroxycoprostanic acid AND/OR 3a,7a,12a-Trihydroxy-5b-cholestan-26-al AND/OR 3alpha,7alpha-dihydroxy-5beta-cholestan-26-oic acid AND/OR 3alpha,7alpha,12alpha-trihydroxy-5beta-cholestan-26-al AND/OR 3alpha,7alpha,24-trihydroxy-5beta-cholestan-26-al AND/OR 7alpha,12alpha,24-trihydroxy-5beta-cholestan-3-one AND/OR 7alpha,25,27-trihydroxycholesterol;

and correlating the identification of the at least 2 biomarkers with laminitis disease status, wherein a statistically relevant level of each of said biomarkers is present in said sample.

The inventors consider that the mere presence of a combination of at least 2 or more of these 5 types of unique metabolites, provided it is a statistically relevant, is very likely to be sufficient to warrant a diagnosis of laminitis even if only at the early stages. Identifying the disease at an early stage may be crucial to enabling recovery of the animal. This is particularly the case where at least 1 of the at least 2 biomarkers is C17 Sphinganine. However, in a preferred embodiment the method may involve identifying a combination of 3, 4 or 5 different metabolic biomarkers in a faecal sample from the subject. The biomarker may be measured by LCMS.

A method for determining the course of progression of laminitis disease in an equine subject, comprising measuring a biomarker selected from: at least 1 biomarker in a faecal sample from the subject, wherein said chemical biomarker is selected from:

C17 Sphinganine;

4-Hydroxyestrone sulfate AND/OR 1-O-alpha-D-Glucopyranosyl-D-mannitol AND/OR Melibiitol AND/OR (1(10)E,6a,9b)-9-(2-Methylpropanoyloxy)-1(10),4,11(13)-germacratrien-12,6-olide AND/OR Ubiquinone-2;

Citronellyl beta-sophoroside AND/OR 1alpha,11alpha,25-trihydroxycholecalciferol AND/OR 1alpha,11beta,25-trihydroxycholecalciferol AND/OR 1alpha,18,25-trihydroxycholecalciferol AND/OR 1alpha,2,25-trihydroxycholecalciferol AND/OR 1alpha,20,25-trihydroxycholecalciferol AND/OR 1alpha,22,25-trihydroxy-20-epicholecalciferol AND/OR 1alpha,23,25-trihydroxycholecalciferol AND/OR 1alpha,24,25-trihydroxycholecalciferol AND/OR 1alpha,25-dihydroxy-10,19-methano-23-oxacholecalciferol AND/OR 1alpha,25-dihydroxy-24a-homo-22-oxa-20-epicholecalciferol AND/OR 1alpha,25-dihydroxy-24a-homo-22-oxacholecalciferol AND/OR 1alpha,25,26-trihydroxycholecalciferol AND/OR 1alpha,26-trihydroxycholecalciferol AND/OR 22,24,25-trihydroxycholecalciferol AND/OR 23,24,25-trihydroxycholecalciferol AND/OR 23,25,26-trihydroxycholecalciferol AND/OR 24,25,26-trihydroxycholecalciferol AND/OR 6,19-epidioxy-1alpha-hydroxy-6,19-dihydrocholecalciferol AND/OR 6,19-epidioxy-25-hydroxy-6,19-dihydrocholecalciferol AND/OR 24-Hydroxycalcitriol AND/OR 3 beta,7 alpha-Dihydroxy-5-cholestenoate AND/OR 3beta,7alpha-dihydroxycholest-5-en-27-oic acid AND/OR 5beta-spirostan-1beta,3alpha-diol AND/OR 7alpha,12alpha,24-trihydroxycholest-4-en-3-one AND/OR 7alpha,12alpha,26-trihydroxycholest-4-en-3-one;

10beta-Hydroxy-6beta-isobutyrylfuranoeremophilane AND/OR 5′-carboxy-alpha-chromanol AND/OR QH2 AND/OR Testolate;

1alpha,19,25-trihydroxy-10,19-dihydrocholecalciferol AND/OR 1alpha,25-dihydroxy-2alpha-hydroxymethyl-19-nor-20-epicholecalciferol AND/OR 1alpha,25-dihydroxy-2alpha-hydroxymethyl-19-norcholecalciferolcholecalciferol AND/OR 1alpha,25-dihydroxy-2beta-hydroxymethyl-19-nor-20-epicholecalciferol AND/OR 1alpha,25-dihydroxy-2beta-hydroxymethyl-19-norcholecalciferol AND/OR 3a,7a-Dihydroxycoprostanic acid AND/OR 3a,7a,12a-Trihydroxy-5b-cholestan-26-al AND/OR 3alpha,7alpha-dihydroxy-5beta-cholestan-26-oic acid AND/OR 3alpha,7alpha,12alpha-trihydroxy-5beta-cholestan-26-al AND/OR 3alpha,7alpha,24-trihydroxy-5beta-cholestan-26-al AND/OR 7alpha,12alpha,24-trihydroxy-5beta-cholestan-3-one AND/OR 7alpha,25,27-trihydroxycholesterol in the faecal sample from said subject at a first time point; measuring said same biomarker in a faecal sample from said patient at a second later time point; and comparing said measurement at said second time point to said measurement at said first time point, wherein a statistically relevant change in concentration in the biomarker indicates progression or regression, respectively, of laminitis in said subject.

The step of correlation between the biomarker and the laminitis disease status in any of the above methods may be provided by a software classification algorithm.

The applicants note that the Fusaria species which produce the mycotoxin fumonisin or FB1 may infect pasture, grasses and cereals, including barley which is a main feed stock for horses. Fumonisin levels are likely to rise during warm damp conditions in spring and autumn when grass is potentially contaminated by fusaria species (and decrease in the winter). This pattern is consistent with the reported incidences of laminitis and in view of the study result, further support that the proposed dual mechanism is the cause of laminitis and that the proposed solutions of the invention are useful.

Following the clear link between toxin presence and disease pathway, detection of such toxin from the outset would therefore have a considerable advantage. The applicant has also therefore sought to prevent problems resulting from FB1 ingestion by further investigating if detection at source is possible and thus providing the opportunity for identification and avoidance of potentially contaminated and thus disease-inducing feed. These investigations have led to a workable assay enabling detection of fumonisin FB1 in a sample e.g. of pasture or other food source as well as from the stool of the animal.

Therefore, in a further aspect of the invention, there is provided a competitive immunoassay to detect the presence of fumonisin. The assays were used to investigate both stool and pasture samples. It was possible to detect fumonisin spiked into both sample types, and “wild type” pasture samples were identified which were detected in the assay. The assay may comprise the features discussed herein below. The invention further relates to a method for determining the presence of mycotoxin fumonisin in an equine food source, or an equine stool sample comprising undertaking a competitive immunoassay comprising immobilised fumonisin and an anti-fumonisin antibody conjugated to HRP wherein competition occurs between immobilised fumonisin and fumonisin in the sample for limited antibody binding sites and the captured antibody is detected: and measuring a signal output, wherein a statistically relevant reduction in the signal output of the sample signifies the presence of fumonisin in the sample. The captured antibody may be detected enzymically with a colorimetric read out such that presence of fumonisin in the sample results in a decrease in the signal generated.

As expected the invention equally concerns a fumonisin B1 antibody, antibody fragment and/or antibody conjugate for use in the identification of fumonisin in an equine food source, or an equine stool sample. Such an antibody, a fumonisin B1 monoclonal antibody, may also be for use in the diagnosis of laminitis. The invention further relates to a dipstick field test incorporating such an immune assay.

Use of such a test will help identify and thus prevent unintended exposure to such toxins in animal feed reducing the laminitic disease risk for animals, such as horses, that are grazing in a new pasture area or otherwise feeding from a new food source that might unknowingly be harmful. The invention may further extend to an immuno-based assay comprising fumonisin B1 antibodies or fumonisin B1 anti-antibodies for use in identification and/or a method of monitoring the progression or regression of laminitis disease in an equine.

In addition, another composition is disclosed for use in the treatment of laminitis which comprises an agent acting to prevent or reduce the production or action of mycotoxin fumonisin B1. Such an agent has specific anti-toxic properties (to enable it to be useful against toxin produced by Fusarium).

In embodiments, it is considered that the agent may be selected from a binder, enzyme and/or absorbent or neutraliser. In one embodiment, the composition may further comprise enzyme-rich malt extracts (ERME) to yield a means by which the action of any residual toxin reaching the gut is prevented. The enhanced effects which may be obtained by this dual therapy are proposed by the mechanisms of action explained by the applicant.

Various further aspects and embodiments of the present invention will be apparent to those skilled in the art in view of the present disclosure. For example, vasculitis is considered an inherent inflammatory condition that is developed during pathogenesis and development of laminitis and thus the applicant considers that the diagnostic methods described herein are relevant to the identification and treatment of vasculitis in horses, hooved animals and animals more generally. The invention therefore extends to medical uses particular to the diagnosis and treatment of vasculitis.

### BRIEF DESCRIPTION

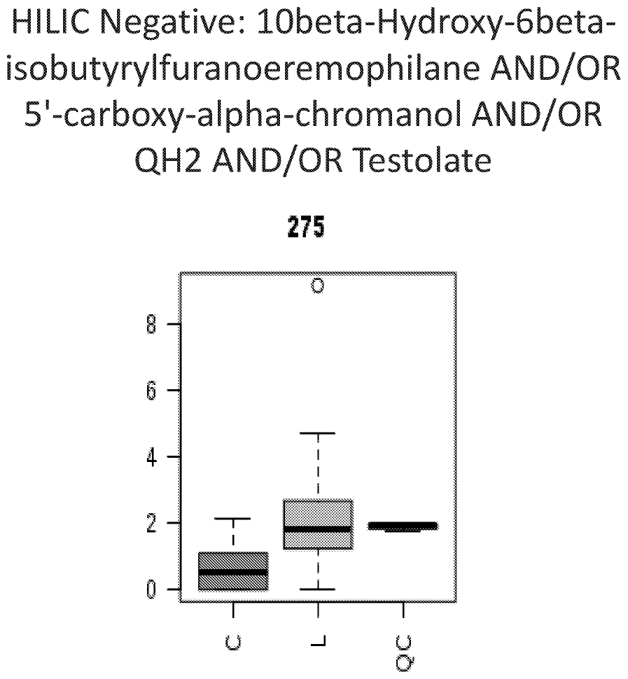

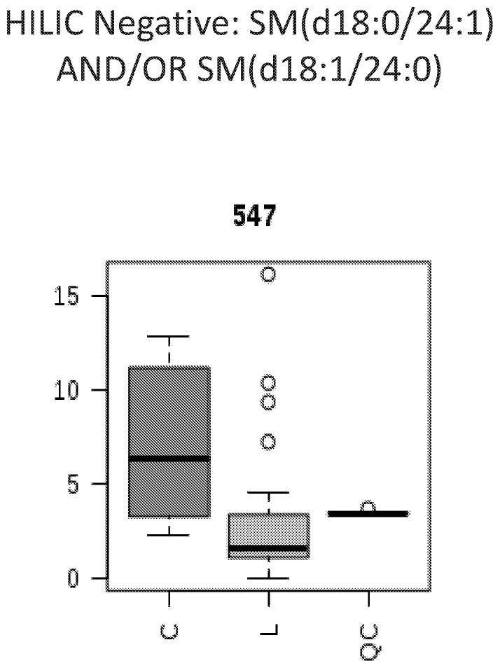

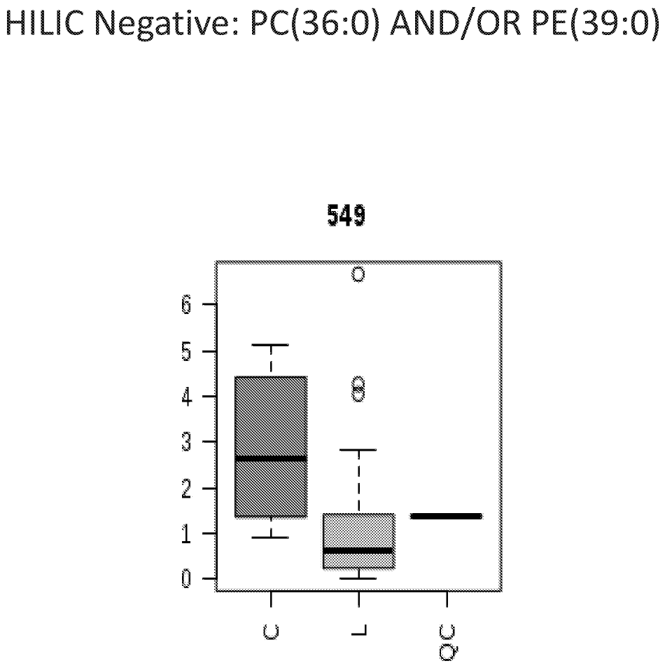

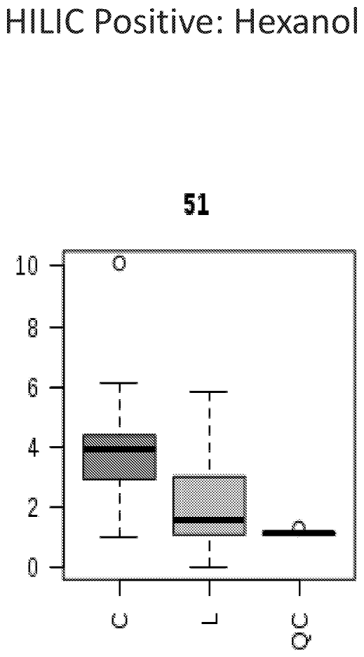

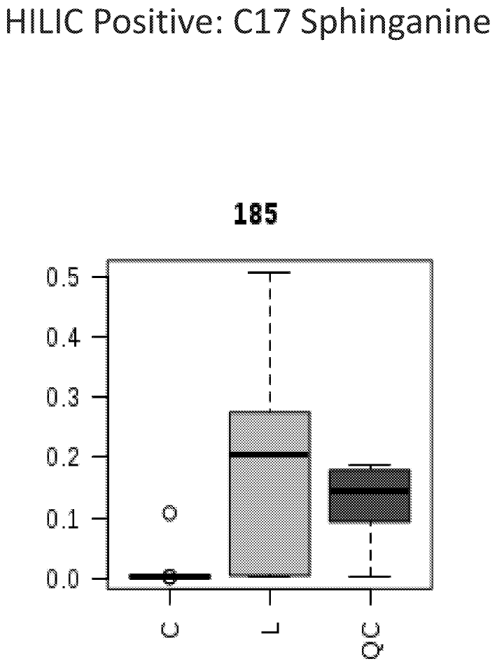

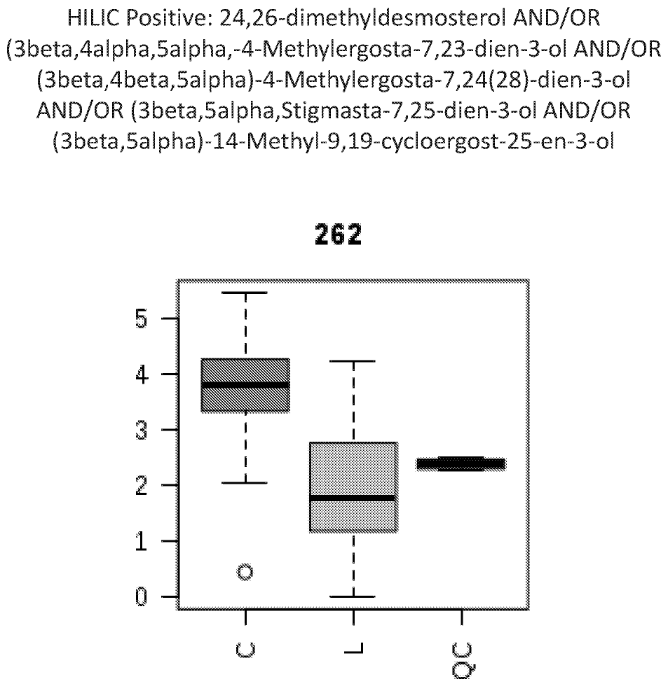

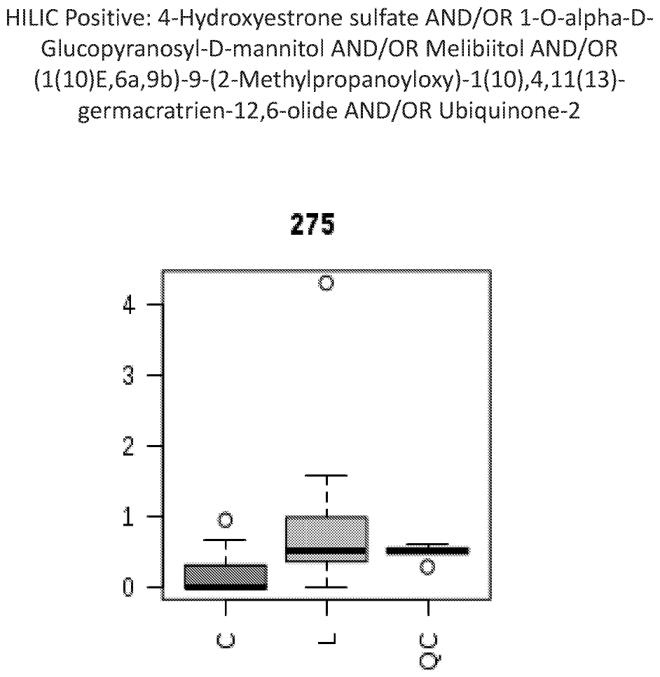

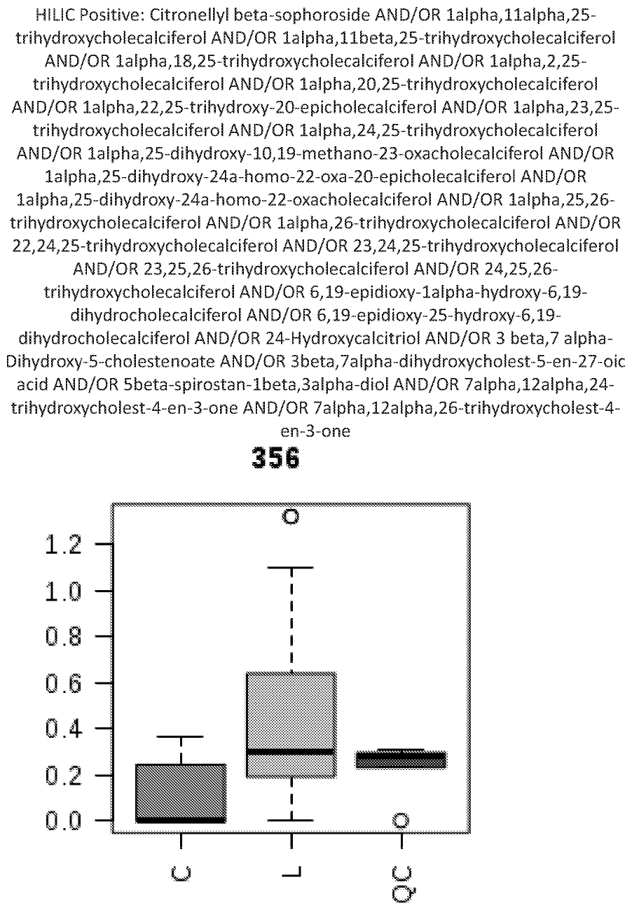

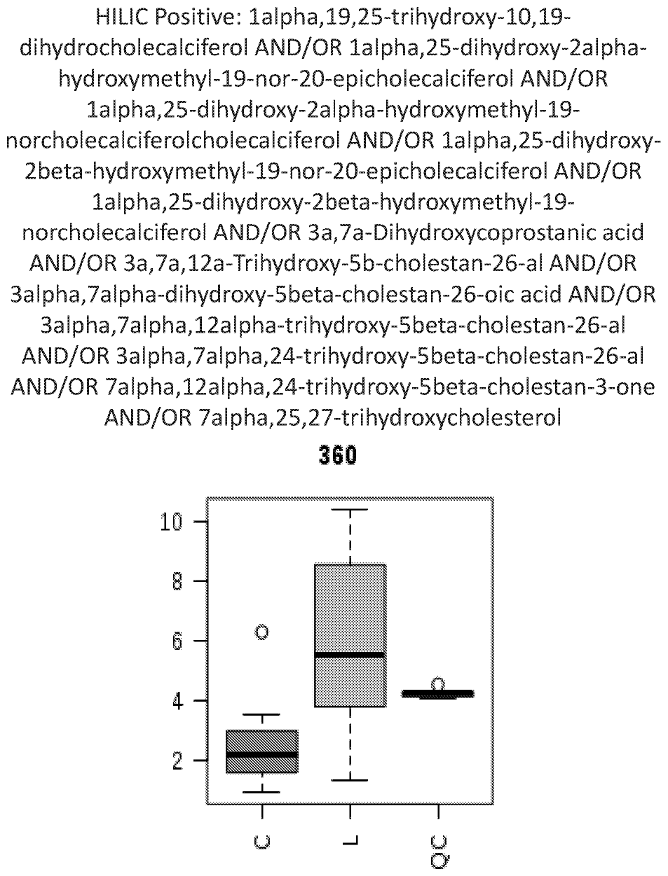

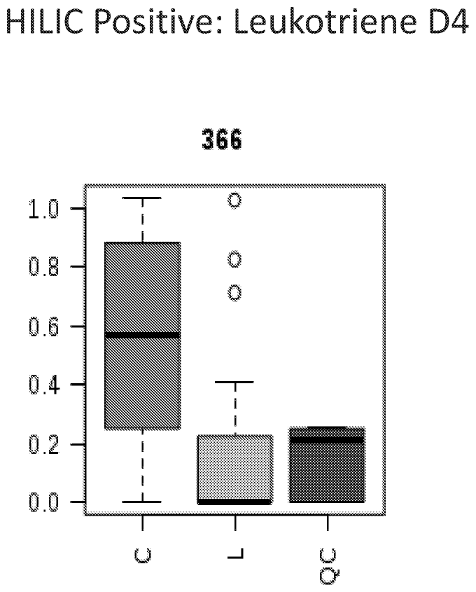

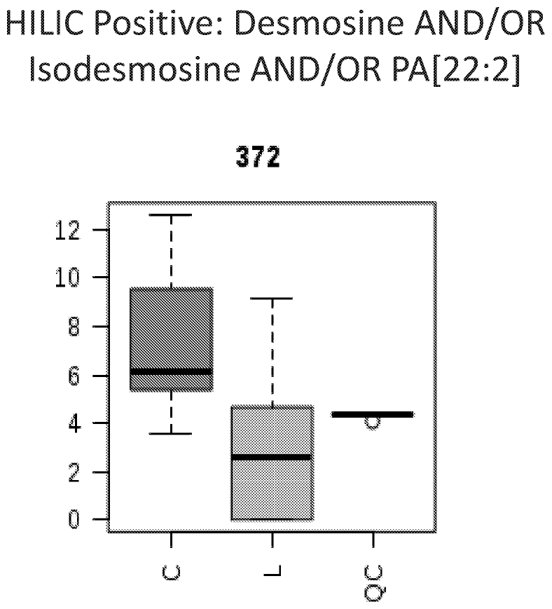

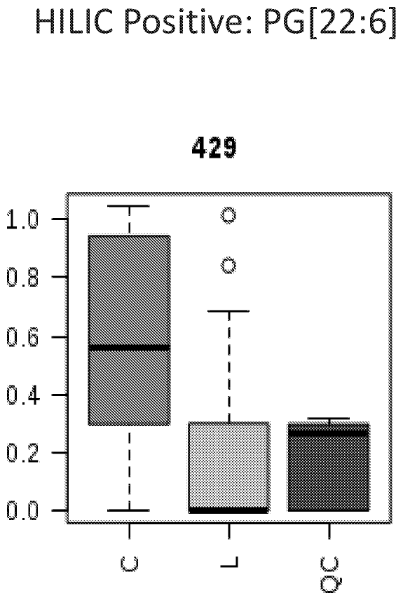

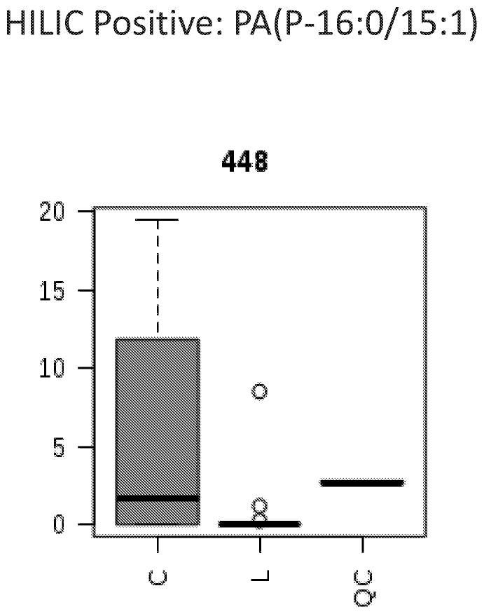

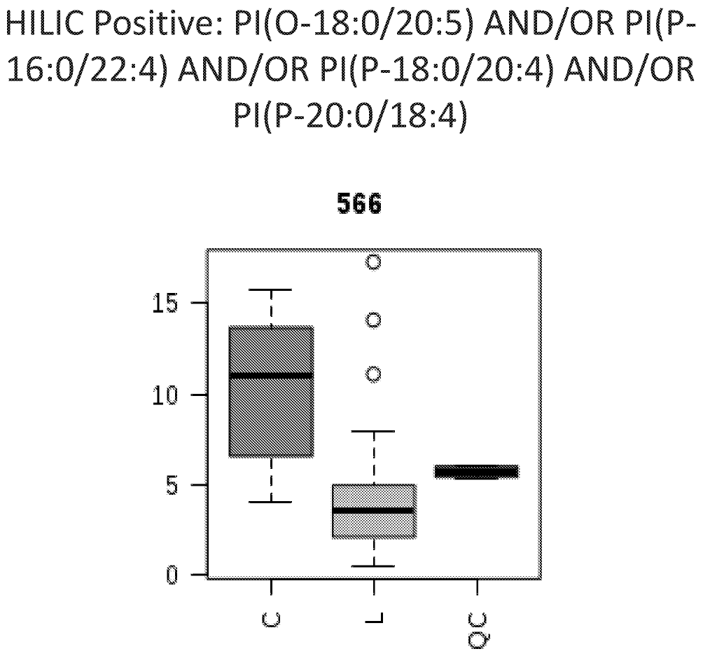

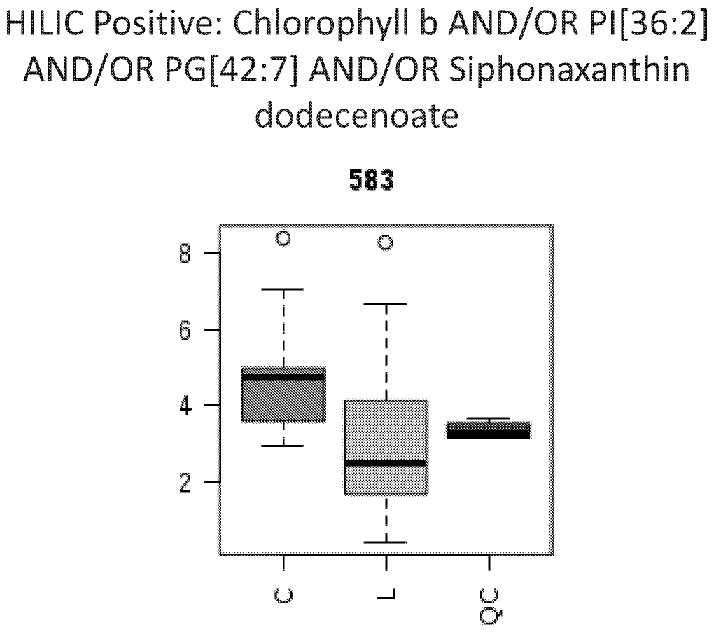

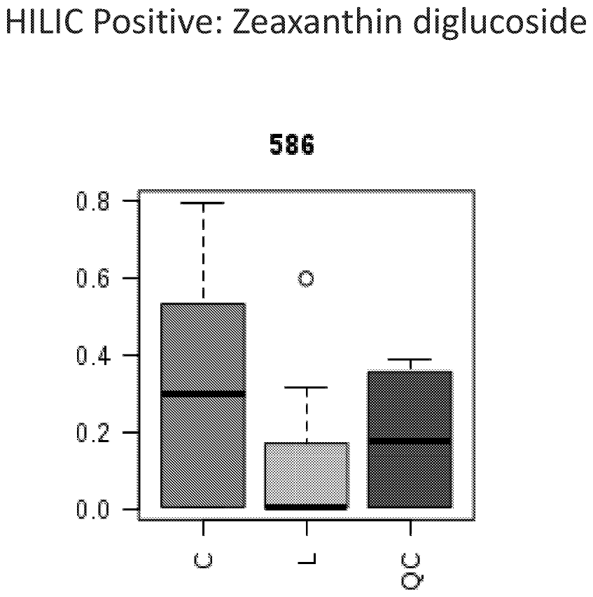

The following figures: FIG. 1 to FIG. 16, provide a graphical illustration of the change in the relevant metabolite identified between control and laminitic horses.

The relevance of the results is described in more detail herein below.

### DETAILED DESCRIPTION

Some authors have hypothesised that further study of the microbiome has the potential to be useful in the diagnosis of equine disease. However, no such study (other than that of the present author as detailed below) has been completed with any useful conclusion to determine how altering the microbiome may be exploited to improve health by of the horse by way of diagnosis and/or treatment. For example, studies testing feeds of exogenous enzymes, such as amylase, to enhance starch production and improve digestion in the microbiome were not deemed conclusively beneficial for improving health.

In their investigations (WO2013/136069) the applicant previously established that the equine microbiome could be altered and improved by providing a feed of several different enzymes, including amylase and fructanase. Such an enzyme combination, particularly when provided from a source of enzyme rich malt extract (ERME), was shown to have a positive effect on the break down plant-based products in the gut. In a study with race horses, a reduction in the abundance of several volatile organic compounds (such as methanol and acetone) in faecal headspace was attributed to the ingestion of ERME; and it was concluded that, in the ERME fed horses, less dietary carbohydrate would reach the gut for microbial fermentation and thus certain chemical by-products could be reduced.

However, the authors believe there to be an unresolved issue in the link between ingestion of excess carbohydrates, metabolic processing of the gut and symptoms of particular equine disease such as laminitis. In particular, the link between a change in the horse microbiome and any subsequent diagnosis of laminitis could not be fully explained by the theoretical rationale or mechanisms proposed in the art. The applicant therefore sought it necessary to further investigate this link.

Changes in the microbiome linking to specific toxic exacerbation and disease have not previously been investigated or evidenced. More particularly, metabolites from an altered gut microbiome had not been identified, let alone reported as relevant, in the context of specific metabolic diseases, such as laminitis. The applicant considered it necessary to investigate the faecal matter chemical profile further with a view to establishing the possibility that specific metabolic products provide useful insight into the causation of laminitis.

The applicant was particularly interested to compare the faecal matter profile of control (C) horses (healthy) and horses with laminitis (L) both to confirm existing suspicions that a change in the microbiome of the gut is implicated in the disease path and whether the presence and/or a significant abundance of specific metabolites reveals specific links to pathogenesis in laminitis.

The present applicant has been able to conclusively demonstrate that particular toxins play an important role in the development of laminitis. The study below has shown, using metabolomics, that the main difference between faeces from control horses and those with laminitis is the presence of a particular chemical metabolite or combination of chemical metabolites that are associated with specific toxins. In combination with the applicant's conclusions (on the relevance of the gut microbial alteration in disease) this work supports that the development of equine disease, such as laminitis, occurs by a simultaneous presence of:

a) an inflammatory state in the gastrointestinal tract, produced by toxins from pathogenic bacteria in when the gut microbiome is altered (due to carbohydrate overload) and

b) the presence of one or more toxins (and in the case of laminitis the Fumonisin toxin from feed contamination with Fusaria).

Such toxic metabolites, whether alone or in combination, therefore serve as useful suitable qualitative and quantitative biomarkers for disease in hooved animals. In particular, the present study concludes that significantly elevated Sphinganine levels provide a useful biomarker for diagnosis of laminitis in hooved animals, particularly horses.

Horses spend large amounts of time grazing on a continuous supply of low-quality nutrients. Bacteria in the hind gut break down the carbohydrates in to short chain fatty acids (SCFAs) which are the main equine energy supply. When conditions change, for example, when horses are kept in stables and fed large amounts of carbohydrate daily, or when they are turned out onto pasture and ingest the new spring grass (rich in complex carbohydrates containing fructose polymers-oligofructose) the microbiome of the gut can be effected. As has been described above, the resulting fermentation in the horse gut appears implicated in the development of laminitis.

If the gut flora alters and pathogenic Gram-negative and/or Gram-positive bacteria result, the pH of the gut falls (acidic conditions). In such a case the cell walls may become more permeable (Moreau M M et al 2014 Vet Microbiol 168 (2-4) 436-441).

The laminae from chronically laminitic horses has been shown to have 100-fold higher levels of more general pathogenic gastrointestinal species such as E. coli and Staphylococcus, suggesting an increased permeability of the gut may have a role to play (Onishi J C et al 2012 Vet Microbiol 158 (3-4) 329-336). Similarly, when laminitis is induced (e.g. by nasogastric dosing of oligofructose) radiolabelled technetium PEG liposomes show systemic inflammation with liposomes accumulating in the colon and lamellar tissue due to increased permeability (Underwood C et al 2015 Vet J 206 (2) 218-225).

However, until the applicant's present work there has been no direct evidence to support a complete pathological mechanism and in particular why inflammation and/or increased permeability in the gut is relevant in diseases of hooved animals.

The applicant's present study reports that the metabolite C17 sphinganine showed a 20-fold excess in laminitic horses. This chemical known as Heptadecasphinganine or (2S,3R)-2-aminoheptadecane-1,3-diol having the formula C₁₇H₃₇NO₂ is therefore considered very significant.

Sphinganine levels are notably raised when the toxin fumonisin B1 (FB1) is ingested. Fumonisin has a structure similar to that of sphinganine and therefore inhibits the incorporation of sphinganine into sphingolipids (inhibition of the enzyme ceramide synthase). Excess levels of sphinganine can therefore build up in vivo. A study (Tadros E M et al 2012 Vet Immunol Immunopath 150 (1-2)90-100) using FB1 on primary isolated epidermal and dermal hoof cells has shown that FB1 is not cytotoxic. However, since it significantly reduces the separation force of explants, this could reduce lamellar integrity and structural strength although the extent of this relationship is not concluded.

On the other hand, the applicant proposes that gut inflammation with an increased permeability of the gut wall and the existence of one or more particular toxins or mycotoxins such as FB1, would mean that such a toxin would rapidly penetrate the systemic circulation with devastating effect. Equines are known to be susceptible to fusaria mycotoxins and other hooved animals may also be affected. Furthermore, in the particular case of laminitis, as evidenced by the present study, such a combination of events appears to lead to a dramatically increased level of a specifically identifiable toxin or combination of toxins or metabolites thereof (Sphinganine).

Horse Study

Sample Preparation

To prepare each sample straw and plant material was removed from fecal matter and the sample was homogenised and extracted in a water/organic solvent solution. The sample was then centrifuged and the clear supernatant was transferred to a UPLC vial.

Assays

Two assays using Ultra performance Liquid Chromatography-Mass Spectrometry were utilised to provide as broad and complete analysis of chemical metabolites as possible.

Each assay operated in positive and negative ion modes: HILIC: Hydrophilic Interaction Chromatography which was optimal for water-soluble polar metabolites and lipidomics which was optimal for lipid metabolites.

The samples were analysed in random order and data analysis undertaken as follows:
Raw data processed applying the software XCMS to generate a data matrix of metabolite features (rows) vs. samples (columns) and filled with chromatographic peak areas
Data filtering: remove all metabolite features with a QC relative standard deviation >20% and not detected in >60% of QC samples
This constructs a processed dataset of high quality for univariate and multivariate analysis
Performed Principal Components Analysis (PCA) to assess data quality and to assess whether there is any biological separation of different classes (time points)
Performed Mann-Whitney U test to identify statistically significant metabolite features with post-hoc tests (p<0.05, FDR-corrected)
Applied PUTMEDID_LCMS software to putatively annotate metabolite features
Where multiple metabolite features were reported for the same metabolite then all metabolite features were deleted except the metabolite feature with the most statistically significant p-value

Boxplots for each metabolite shown to be statistically significant were constructed and are shown in FIGS. 1 to 16.

TABLE 1
Results

| | | | Fold | | | | Putative | |
|---|---|---|---|---|---|---|---|---|
| | | | Change | | | Retention | Annotated | Metabolite |
| Idx | p. value | FDR | (C/L) | Assay | m/z | time (s) | Metabolites | Class |
| 372 | 0.000023 | 0.0069 | 2.57 | HILIC | 565.2484 | 43.9 | Desmosine | Mixed class |
| | | | | positive | | | AND/OR | |
| | | | | | | | Isodesmosine | |
| | | | | | | | AND/OR PA[22:2] | |
| 360 | 0.000115 | 0.0100 | 0.41 | HILIC | 552.2688 | 43.8 | 1alpha,19,25- | Sterol and |
| | | | | positive | | | trihydroxy-10,19- | steroid |
| | | | | | | | dihydrocholecalciferol | metabolism |
| | | | | | | | AND/OR | |
| | | | | | | | 1alpha,25- | |
| | | | | | | | dihydroxy-2alpha- | |
| | | | | | | | hydroxymethyl- | |
| | | | | | | | 19-nor-20- | |
| | | | | | | | epicholecalciferol | |
| | | | | | | | AND/OR | |
| | | | | | | | 1alpha,25- | |
| | | | | | | | dihydroxy-2alpha- | |
| | | | | | | | hydroxymethyl- | |
| | | | | | | | 19- | |
| | | | | | | | norcholecalciferol | |
| | | | | | | | cholecalciferol | |
| | | | | | | | AND/OR | |
| | | | | | | | 1alpha,25- | |
| | | | | | | | dihydroxy-2beta- | |
| | | | | | | | hydroxymethyl- | |
| | | | | | | | 19-nor-20- | |
| | | | | | | | epicholecalciferol | |
| | | | | | | | AND/OR | |
| | | | | | | | 1alpha,25- | |
| | | | | | | | dihydroxy-2beta- | |
| | | | | | | | hydroxymethyl- | |
| | | | | | | | 19- | |
| | | | | | | | norcholecalciferol | |
| | | | | | | | AND/OR 3a,7a- | |
| | | | | | | | Dihydroxycoprostanic | |
| | | | | | | | acid AND/OR | |
| | | | | | | | 3a,7a,12a- | |
| | | | | | | | Trihydroxy-5b- | |
| | | | | | | | cholestan-26-al | |
| | | | | | | | AND/OR | |
| | | | | | | | 3alpha,7alpha- | |
| | | | | | | | dihydroxy-5beta- | |
| | | | | | | | cholestan-26-oic | |
| | | | | | | | acid AND/OR | |
| | | | | | | | 3alpha,7alpha,12alpha- | |
| | | | | | | | trihydroxy- | |
| | | | | | | | 5beta-cholestan- | |
| | | | | | | | 26-al AND/OR | |
| | | | | | | | 3alpha,7alpha,24- | |
| | | | | | | | trihydroxy-5beta- | |
| | | | | | | | cholestan-26-al | |
| | | | | | | | AND/OR | |
| | | | | | | | 7alpha,12alpha,24- | |
| | | | | | | | trihydroxy-5beta- | |
| | | | | | | | cholestan-3-one | |
| | | | | | | | AND/OR | |
| | | | | | | | 7alpha,25,27- | |
| | | | | | | | trihydroxycholestrol | |
| 366 | 0.000151 | 0.0100 | 3.06 | HILIC | 555.2252 | 36.8 | Leukotriene D4 | Leukotrienes |
| | | | | positive | | | | and |
| | | | | | | | | prostaglandins |
| 566 | 0.000066 | 0.0100 | 2.18 | HILIC | 893.5485 | 36.7 | PI(O-18:0/20:5) | Glycerophospholipids |
| | | | | positive | | | AND/OR | |
| | | | | | | | PI(P-16:0/22:4) | |
| | | | | | | | AND/OR | |
| | | | | | | | PI(P-18:0/20:4) | |
| | | | | | | | AND/OR | |
| | | | | | | | PI(P-20:0/18:4) | |
| 262 | 0.000223 | 0.0120 | 1.82 | HILIC | 413.3775 | 39.9 | 24,26- | Sterol and |
| | | | | positive | | | dimethyldesmosterol | steroid |
| | | | | | | | AND/OR | metabolism |
| | | | | | | | (3beta,4alpha,5alpha,- | |
| | | | | | | | 4-Methylergosta- | |
| | | | | | | | 7,23-dien-3-ol | |
| | | | | | | | AND/OR | |
| | | | | | | | (3beta,4beta,5alpha)- | |
| | | | | | | | 4-Methylergosta- | |
| | | | | | | | 7,24(28)-dien-3-ol | |
| | | | | | | | AND/OR | |
| | | | | | | | (3beta,5alpha,Stigmasta- | |
| | | | | | | | 7,25-dien- | |
| | | | | | | | 3-ol AND/OR | |
| | | | | | | | (3beta,5alpha)-14- | |
| | | | | | | | Methyl-9,19- | |
| | | | | | | | cycloergost-25-en- | |
| | | | | | | | 3-ol | |
| 185 | 0.000368 | 0.0182 | 0.05 | HILIC | 288.2908 | 158.4 | C17 Sphinganine | Ceramides and |
| | | | | positive | | | | sphingolipids |
| 547 | 0.000223 | 0.0241 | 2.22 | HILIC | 871.6464 | 38.0 | SM(d18:0/24:1) | Ceramides and |
| | | | | negative | | | AND/OR | sphingolipids |
| | | | | | | | SM(d18:1/24:0) | |
| 549 | 0.000172 | 0.0241 | 2.33 | HILIC | 872.4855 | 37.7 | PC(36:0) AND/OR | Glycerophospholipids |
| | | | | negative | | | PE(39:0) | |
| 275 | 0.000132 | 0.0241 | 0.29 | HILIC | 435.1083 | 42.8 | 10beta-Hydroxy- | Mixed class |
| | | | | negative | | | 6beta- | |
| | | | | | | | isobutyrylfuranoer | |
| | | | | | | | emophilane | |
| | | | | | | | AND/OR 5′- | |
| | | | | | | | carboxy-alpha- | |
| | | | | | | | chromanol | |
| | | | | | | | AND/OR QH2 | |
| | | | | | | | AND/OR Testolate | |
| 51 | 0.001047 | 0.0296 | 1.86 | HILIC | 141.0670 | 317.1 | Hexanol | Fatty acid and |
| | | | | positive | | | | oxidised fatty |
| | | | | | | | | acid |
| 275 | 0.000937 | 0.0296 | 0.26 | HILIC | 435.1083 | 42.8 | 4-Hydroxyestrone | Mixed class |
| | | | | positive | | | sulfate AND/OR 1- | |
| | | | | | | | O-alpha-D- | |
| | | | | | | | Glucopyranosyl-D- | |
| | | | | | | | mannitol AND/OR | |
| | | | | | | | Melibiitol AND/OR | |
| | | | | | | | (1(10)E,6a,9b)-9- | |
| | | | | | | | (2-Methylpropanoyloxy)- | |
| | | | | | | | 1(10),4,11(13)- | |
| | | | | | | | germacratrien- | |
| | | | | | | | 12,6-olide | |
| | | | | | | | AND/OR | |
| | | | | | | | Ubiquinone-2 | |
| 356 | 0.001047 | 0.0296 | 0.28 | HILIC | 550.2532 | 42.8 | Citronellyl beta- | Mixed class |
| | | | | positive | | | sophoroside | |
| | | | | | | | AND/OR | |
| | | | | | | | 1alpha,11alpha,25- | |
| | | | | | | | trihydroxycholecalciferol | |
| | | | | | | | AND/OR | |
| | | | | | | | 1alpha,11beta,25- | |
| | | | | | | | trihydroxycholecalciferol | |
| | | | | | | | AND/OR | |
| | | | | | | | 1alpha,18,25- | |
| | | | | | | | trihydroxycholecalciferol | |
| | | | | | | | AND/OR | |
| | | | | | | | 1alpha,2,25- | |
| | | | | | | | trihydroxycholecalciferol | |
| | | | | | | | AND/OR | |
| | | | | | | | 1alpha,20,25- | |
| | | | | | | | trihydroxycholecalciferol | |
| | | | | | | | AND/OR | |
| | | | | | | | 1alpha,22,25- | |
| | | | | | | | trihydroxy-20- | |
| | | | | | | | epicholecalciferol | |
| | | | | | | | AND/OR | |
| | | | | | | | 1alpha,23,25- | |
| | | | | | | | trihydroxycholecalciferol | |
| | | | | | | | AND/OR | |
| | | | | | | | 1alpha,24,25- | |
| | | | | | | | trihydroxycholecalciferol | |
| | | | | | | | AND/OR | |
| | | | | | | | 1alpha,25- | |
| | | | | | | | dihydroxy-10,19- | |
| | | | | | | | methano-23- | |
| | | | | | | | oxacholecalciferol | |
| | | | | | | | AND/OR | |
| | | | | | | | 1alpha,25- | |
| | | | | | | | dihydroxy-24a- | |
| | | | | | | | homo-22-oxa-20- | |
| | | | | | | | epicholecalciferol | |
| | | | | | | | AND/OR | |
| | | | | | | | 1alpha,25- | |
| | | | | | | | dihydroxy-24a- | |
| | | | | | | | homo-22- | |
| | | | | | | | oxacholecalciferol | |
| | | | | | | | AND/OR | |
| | | | | | | | 1alpha,25,26- | |
| | | | | | | | trihydroxycholecalciferol | |
| | | | | | | | AND/OR | |
| | | | | | | | 1alpha,26- | |
| | | | | | | | trihydroxycholecalciferol | |
| | | | | | | | AND/OR | |
| | | | | | | | 22,24,25- | |
| | | | | | | | trihydroxycholecalciferol | |
| | | | | | | | AND/OR | |
| | | | | | | | 23,24,25- | |
| | | | | | | | trihydroxycholecalciferol | |
| | | | | | | | AND/OR | |
| | | | | | | | 23,25,26- | |
| | | | | | | | trihydroxycholecalciferol | |
| | | | | | | | AND/OR | |
| | | | | | | | 24,25,26- | |
| | | | | | | | trihydroxycholecalciferol | |
| | | | | | | | AND/OR | |
| | | | | | | | 6,19-epidioxy- | |
| | | | | | | | 1alpha-hydroxy- | |
| | | | | | | | 6,19- | |
| | | | | | | | dihydrocholecalciferol | |
| | | | | | | | AND/OR 6,19- | |
| | | | | | | | epidioxy-25- | |
| | | | | | | | hydroxy-6,19- | |
| | | | | | | | dihydrocholecalciferol | |
| | | | | | | | AND/OR 24- | |
| | | | | | | | Hydroxycalcitriol | |
| | | | | | | | AND/OR 3 beta,7 | |
| | | | | | | | alpha-Dihydroxy- | |
| | | | | | | | 5-cholestenoate | |
| | | | | | | | AND/OR | |
| | | | | | | | 3beta,7alpha- | |
| | | | | | | | dihydroxycholest- | |
| | | | | | | | 5-en-27-oic acid | |
| | | | | | | | AND/OR 5beta- | |
| | | | | | | | spirostan- | |
| | | | | | | | 1beta,3alpha-diol | |
| | | | | | | | AND/OR | |
| | | | | | | | 7alpha,12alpha,24- | |
| | | | | | | | trihydroxycholest- | |
| | | | | | | | 4-en-3-one | |
| | | | | | | | AND/OR | |
| | | | | | | | 7alpha,12alpha,26- | |
| | | | | | | | trihydroxycholest- | |
| | | | | | | | 4-en-3-one | |
| 429 | 0.000748 | 0.0296 | 2.92 | HILIC | 615.2440 | 36.7 | PG[22:6] | Glycerophospholipids |
| | | | | positive | | | | |
| 583 | 0.001450 | 0.0359 | 1.55 | HILIC | 909.5299 | 36.7 | Chlorophyll b | Mixed class |
| | | | | positive | | | AND/OR PI[36:2] | |
| | | | | | | | AND/OR PG[42:7] | |
| | | | | | | | AND/OR | |
| | | | | | | | Siphonaxanthin | |
| | | | | | | | dodecenoate | |
| 586 | 0.001613 | 0.0383 | 3.78 | HILIC | 915.5238 | 36.5 | Zeaxanthin | Carotenoid |
| | | | | positive | | | diglucoside | biosynthesis |
| 448 | 0.002201 | 0.0484 | 11.94 | HILIC | 639.4342 | 40.7 | PA(P-16:0/15:1) | Glycerophospholipids |
| | | | | positive | | | | |

The table shows that 16 unique metabolite features showed a statistically significant change in concentration between control and laminitis horses (as defined in faecal matter) and as such theses metabolites are putative metabolic biomarkers diagnostic of laminitis in horses.

The graphs illustrating whether a statistically relevant change takes place are shown below in the figures from 1 to 16.

5 unique metabolite features showed a higher concentration in laminitis horse faecal matter compared to control horses. Further, a combination of such biomarkers is a powerful indication of laminitis pathogenesis.

Listed in order of greatest fold change these are:

C17 Sphinganine
4-Hydroxyestrone sulfate AND/OR 1-O-alpha-D-Glucopyranosyl-D-mannitol AND/OR Melibiitol
AND/OR (1(10)E,6a,9b)-9-(2-Methylpropanoyloxy)-1(10),4,11(13)-germacratrien-12,6-olide AND/OR
Ubiquinone-2
Citronellyl beta-sophoroside AND/OR 1alpha,11alpha,25-trihydroxycholecalciferol AND/OR
1alpha,11beta,25-trihydroxycholecalciferol AND/OR 1alpha,18,25-trihydroxycholecalciferol AND/OR
1alpha,2,25-trihydroxycholecalciferol AND/OR 1alpha,20,25-trihydroxycholecalciferol AND/OR
1alpha,22,25-trihydroxy-20-epicholecalciferol AND/OR 1alpha,23,25-trihydroxycholecalciferol
AND/OR 1alpha,24,25-trihydroxycholecalciferol AND/OR 1alpha,25-dihydroxy-10,19-methano-23-
oxacholecalciferol AND/OR 1alpha,25-dihydroxy-24a-homo-22-oxa-20-epicholecalciferol AND/OR
1alpha,25-dihydroxy-24a-homo-22-oxacholecalciferol AND/OR 1alpha,25,26-trihydroxycholecalciferol
AND/OR 1alpha,26-trihydroxycholecalciferol AND/OR 22,24,25-trihydroxycholecalciferol AND/OR
23,24,25-trihydroxycholecalciferol AND/OR 23,25,26-trihydroxycholecalciferol AND/OR 24,25,26-
trihydroxycholecalciferol AND/OR 6,19-epidioxy-1alpha-hydroxy-6,19-dihydrocholecalciferol AND/OR
6,19-epidioxy-25-hydroxy-6,19-dihydrocholecalciferol AND/OR 24-Hydroxycalcitriol AND/OR 3 beta,7
alpha-Dihydroxy-5-cholestenoate AND/OR 3beta,7alpha-dihydroxycholest-5-en-27-oic acid AND/OR
5beta-spirostan-1beta,3alpha-diol AND/OR 7alpha,12alpha,24-trihydroxycholest-4-en-3-one AND/OR
7alpha,12alpha,26-trihydroxycholest-4-en-3-one
10beta-Hydroxy-6beta-isobutyrylfuranoeremophilane AND/OR 5′-carboxy-alpha-chromanol AND/OR
QH2 AND/OR Testolate
1alpha,19,25-trihydroxy-10,19-dihydrocholecalciferol AND/OR 1alpha,25-dihydroxy-2alpha-
hydroxymethyl-19-nor-20-epicholecalciferol AND/OR 1alpha,25-dihydroxy-2alpha-hydroxymethyl-19-
norcholecalciferolcholecalciferol AND/OR 1alpha,25-dihydroxy-2beta-hydroxymethyl-19-nor-20-
epicholecalciferol AND/OR 1alpha,25-dihydroxy-2beta-hydroxymethyl-19-norcholecalciferol AND/OR
3a,7a-Dihydroxycoprostanic acid AND/OR 3a,7a,12a-Trihydroxy-5b-cholestan-26-al AND/OR
3alpha,7alpha-dihydroxy-5beta-cholestan-26-oic acid AND/OR 3alpha,7alpha,12alpha-trihydroxy-
5beta-cholestan-26-al AND/OR 3alpha,7alpha,24-trihydroxy-5beta-cholestan-26-al AND/OR
7alpha,12alpha,24-trihydroxy-5beta-cholestan-3-one AND/OR 7alpha,25,27-trihydroxycholesterol

Of all the metabolites, C17 Sphinganine showed the largest change, where the Laminitis value (L) which corresponds to 20 times the control value (C). The fold changes are defined directly in terms of ratios. If the control value is C and the Laminitis value L, the fold change is defined as C/L. In this case L=20 and C=1 and thus 1/20=0.05.

In this case such a fold change represents a biomarker at a significantly higher concentration in the ‘disease’ samples compared to the ‘control’.

The applicant believes that these results evidence a clear link between diagnoses of laminitis and chemical metabolite profiling difference in a horse's faecal matter as compared to a healthy animal.

In particular, where at least one or a combination of the above 5 biomarkers is present in that profile, laminitis is highly likely to be indicated. More particularly, where the concentration of a particular metabolite is found to be above statistically above the control threshold, laminitis is likely to be indicated.

The presence of C17 Sphinganine, in particular would appear marked, relative to the healthy animal control. As such a statistically relevant fold change in the concentration of C17 Sphinganine would appear to indicate the horse is suffering from laminitis.

As discussed at length, fusaria species producing the mycotoxin FB1 typically infect grasses and cereals, including barley especially during the warm, damp seasons. The applicant notes that methods of detection are therefore paramount to positive avoidance of toxin-contaminated feed and prevention of disease. In this regard the applicant has developed a method to enable such detection by formulating as assay and then testing several samples.

Immunoassay Method for Detection

The applicant developed two forms of working immunoassay for testing for FB1 toxin in samples; low and high sensitivity. In each, a competitive immunoassay was used in which with fumonisin immobilised on the plate via streptavidin and a commercially available anti-fumonisin antibody which was conjugated to HRP.

Competition occurred between immobilised fumonisin and fumonisin in the sample for the limited antibody binding sites.

Captured antibody was detected enzymically with a colorimetric read out such that presence of fumonisin in the sample resulted in a decrease in the signal generated.

The assays were used to investigate both stool and pasture samples.

It was possible to detect fumonisin spiked into both sample types, and “wild type” pasture samples were identified which were detected in the assay.

An example protocol was followed, as provided below:

Lower Sensitivity:

50 μl of 20 ng/ml Fumonisin Biotin Conjugate (low molecular weight of FB1 necessitated that it be conjugated to a protein carrier) was added to each well of the Streptavidin coated microtiter plate and incubated for 1 hour at 37° C. and then washed three times with Assay Buffer 47. Next, 50 μl of unconjugated Fumonisin ranging from 1000 ng/ml to 0 ng/ml was added across the plate in its respective row and 50 μl of 20 ng/ml HRP Conjugate was added to every well. The plate was incubated for 1 hour at 37° C., 450 rpm and washed three times with Assay Buffer 47. 100 μl of Sure Blue Reserve TMB was added to each well and colour was left to develop for 10 minutes in the dark at room temperature. Colour development was stopped by addition of 100 μL per well of 1M HCl.

The plate was read at 450 nm.

Higher Sensitivity:

50 μl of 0.5 ng/ml Fumonisin Biotin Conjugate was added to each well of the Streptavidin coated microtiter plate and incubated for 1 hour at 37° C. and then washed three times with Assay Buffer 47. Next, 50 μl of unconjugated Fumonisin ranging from 25 ng/ml to 0 ng/ml was added across the plate in its respective row and 50 μl of 100 ng/ml HRP Conjugate was added to every well. The plate was incubated for 1 hour at 37° C., 450 rpm and washed three times with Assay Buffer 47. 100 μl of Sure Blue Reserve TMB was added to each well and colour was left to develop for 10 minutes in the dark at room temperature. Colour development was stopped by addition of 100 μL per well of 1M HCl.

The plate was read at 450 nm.

Results are provided below:

| Unconjugated | | | | | | | | | |
|---|---|---|---|---|---|---|---|---|---|
| Fumonisn | | | | | | | | | |
| (ng/ml) | Standard | | | Samples | | | | | |
| 0 | 1.0222 | 1.1803 | 0.8928 | 1.2566 | 1.1644 | 0.9873 | 1.1555 | | |
| 0.1 | 0.8377 | 0.8679 | 0.7451 | 0.1649 | 0.1772 | 0.1603 | 0.1729 | | |
| 0.5 | 0.4225 | 0.5032 | 0.4498 | 0.5804 | 0.5798 | 0.5235 | 0.5238 | | |
| 1 | 0.2999 | 0.2905 | 0.3089 | 0.2512 | 0.2683 | 0.2327 | 0.2498 | | |
| 5 | 0.1013 | 0.0996 | 0.0977 | 1.0725 | 1.0039 | 1.0032 | 0.9807 | | |
| 10 | 0.0674 | 0.0732 | 0.0692 | 0.1459 | 0.1477 | 0.1468 | 0.1376 | | |
| 25 | 0.0533 | 0.0538 | 0.0538 | 1.2907 | 1.3550 | 1.4085 | 1.5527 | | |
| 50 | 0.0465 | 0.0461 | 0.0462 | 0.0913 | 0.1310 | 0.1071 | 0.1126 | Known negative | |
| | 50 μl 0.5 ng/ml Biotin Fumonisin | | | | | | | Spiked | |
| | | | | | | | | positive | |
| | 50 μl 100 ng/ml HRP CJ2 | | | | | | | | |
| 0.001 | 1.0318 | 0.143989 | 13.96% | | 1.1410 | 0.112168 | 9.83% | FBL1 | |
| 0.1 | 0.8169 | 0.063988 | 7.83% | | 0.1688 | 0.007634 | 4.52% | FBL1 | 0.9721 |
| | | | | | | | | SPIKED | |
| | | | | | | | | @5 ng/ml | |
| 0.5 | 0.4585 | 0.041047 | 8.95% | | 0.5519 | 0.032593 | 5.91% | NTS3 | |
| 1 | 0.2998 | 0.009201 | 3.07% | | 0.2505 | 0.014545 | 5.81% | NTS3 | 0.3014 |
| | | | | | | | | SPIKED | |
| | | | | | | | | @5 ng/ml | |
| 5 | 0.0995 | 0.001801 | 1.81% | | 1.0151 | 0.039771 | 3.92% | SS3 | |
| 10 | 0.0699 | 0.002969 | 4.25% | | 0.1445 | 0.004658 | 3.22% | SS3 | 0.8706 |
| | | | | | | | | SPIKED | |
| | | | | | | | | @5 ng/ml | |
| 25 | 0.0536 | 0.000289 | 0.54% | | 1.4017 | 0.111578 | 7.96% | SS1 | |
| 50 | 0.0463 | 0.000208 | 0.45% | | 0.1105 | 0.016379 | 14.82% | SS1 | 1.2912 |
| | | | | | | | | SPIKED | |
| | | | | | | | | @5 ng/ml | |

Such assays support the proof of concept that such a methodology could be used in field test and thus may be optimised for a quicker result. For example, a typical dipstick assay would use antibody conjugated to coloured particles such as colloidal gold or latex, but the conjugates generated for the ELISA in the above tests could use a nitrocellulose membrane solid phase to produce a faster qualitative version of the assay. However, the applicants have demonstrated how to immobilise fumonisin on the membrane and get a functioning assay by immobilising in a discrete spot and the sample and antibody-conjugate are dropped onto the surface. After a 10 minute incubation the membrane is washed and then placed into a precipitating substrate. After a further 10 minutes the membrane is washed again and the results can be seen as a purple spot for a negative which is abolished with a positive.

Thus, a field test comprising an immuno-based assay system incorporating FB1 antibodies provides a clear way of identifying when the FB1 toxin is present in field samples, such as pasture, feed or stool samples. In particular, this testing demonstrates that the fumonisin can be immobilised on a membrane and retain activity and that the antibody affinity is such that a rapid test for dipstick field testing would be feasible and practically able to be optimised.

Antibodies to FB1 have been made; for example, rapid and efficient production of polyclonal antibodies to FB1 and its analogues by using a conjugate of FB1-CT as the immunogen, as described in https://www.ncbi.nlm.nih.gov/pmc/articles/PMC195187/pdf/aem00042-0189.pdf.

However, they have never been used for the present purposes.

Thus, it is entirely plausible that antibodies to this particular toxin FB1 could be made and thus in the wake of the findings of the present applicant be used in the novel detection/prevention.

Treatment and Prevention of Laminitis

In addition, a further aspect of the invention extends to formulations and compositions for use treating laminitis comprising at least one antifungal agent or an anti-toxin agent acting to neutralise the effects of fumonisin B1. This may be in the form of an agent which prevents or reduces the action of such a toxin when in the gut. Such an agent may therefore substantially neutralise the toxin's effect to prevent or treat laminitis. Such agents may include binders, absorbents, probiotics and/or enzymes.

In particular a binder may be a polymer, such as poly (acrylamide-co-ethylene glycol-dimethacrylate) PA-EGDMA, or biopolymer such as beta-D-glucan based polymer. Absorbents such as cellulose or nano cellulose may be coated with a free fatty acid (Lauric acid, alpha linoleic acid, oleic acid and palmitic acid, for example) may be utilised to enhance the absorbent character of the agent.

Certain probiotic bacteria including lactic acid bacteria may be utilised to provide a protective effect to guard against the toxic effect of FB1 by upregulating existing endogenous enzymatic activity that may serve to reduce the amount of toxin that is absorbed by the gut.

Exogenous enzymes capable of breaking down FB1 in to a neutralised non-toxic metabolite form may be useful in some embodiments. Such enzymes could be useful where identified to be responsible for the detoxification such as an esterase, which would cleave the ester groups from fumonisin rendering it non-toxic.

Further, the above-described compositions having an anti-toxin effect may be enhanced further by the presence of an enzyme rich malt extract, such as ERME. The applicant proposes that reducing gut inflammation and permeability of the gut wall would dramatically reduce the capability of fusaria mycotoxin to penetrate the systemic circulation in the typical way. The applicant's work in this regard highlighting the mechanism of the disease strongly suggests that such a combination treatment would be more effective than an anti-toxin alone.

The use of an ERME formulation in combination with a medicament which actively neutralises the effect of such a toxin would appear to be an effective dual therapy for preventing and treating laminitis due to the specific mechanism of the disease. In particular, the presence of ERME in the combination product may greatly reduce the damaging nature of any toxin ingested, whereas the effect of the ingestion of such a toxin is typically exacerbated by the typical diet. In such a case the fusaria or other toxin will mostly pass through the digestive system, with limited systemic circulation, minimising the biological effect at the hoof or elsewhere.

Thus, application of a diagnostic tool to identify laminitis followed by a treatment of therapeutically effective formulation is a very useful combination to tackle the aforementioned technical problems.

By combining such prevention methods with assays or diagnostic tools and treatment possibilities, the ability to prevent and/or treat this now well understood toxin-led condition of laminitis improves substantially.

All documents mentioned in this specification are incorporated herein by reference in their entirety.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described. Various further aspects and embodiments of the present invention will be apparent to those skilled in the art in view of the present disclosure. While the invention has been described herein in reference to specific aspects, features and illustrative embodiments of the invention, it will be appreciated that the utility of the invention is not thus limited, but rather extends to and encompasses variations, modifications and alternative embodiments, as will be understood by those of ordinary skill in the field.

1. A method for qualifying equine laminitis disease status in a subject comprising:
measuring at least 1 biomarker in a faecal sample from the subject, wherein said chemical biomarker is selected from:
Heptadecasphinganine (2S,3R)-2-aminoheptadecane-1,3-diol) “C17 Sphinganine”;
4-Hydroxyestrone sulfate AND/OR 1-O-alpha-D-Glucopyranosyl-D-mannitol AND/OR Melibiitol AND/OR (1(10)E,6a,9b)-9-(2-Methylpropanoyloxy)-1(10),4,11(13)-germacratrien-12,6-olide AND/OR Ubiquinone-2;
Citronellyl beta-sophoroside AND/OR 1alpha,11alpha,25-trihydroxycholecalciferol AND/OR 1alpha,11beta,25-trihydroxycholecalciferol AND/OR 1alpha,18,25-trihydroxycholecalciferol AND/OR 1alpha,2,25-trihydroxycholecalciferol AND/OR 1alpha,20,25-trihydroxycholecalciferol AND/OR 1alpha,22,25-trihydroxy-20-epicholecalciferol AND/OR 1alpha,23,25-trihydroxycholecalciferol AND/OR 1alpha,24,25-trihydroxycholecalciferol AND/OR 1alpha,25-dihydroxy-10,19-methano-23-oxacholecalciferol AND/OR 1alpha,25-dihydroxy-24a-homo-22-oxa-20-epicholecalciferol AND/OR 1alpha,25-dihydroxy-24a-homo-22-oxacholecalciferol AND/OR 1alpha,25,26-trihydroxycholecalciferol AND/OR 1alpha,26-trihydroxycholecalciferol AND/OR 22,24,25-trihydroxycholecalciferol AND/OR 23,24,25-trihydroxycholecalciferol AND/OR 23,25,26-trihydroxycholecalciferol AND/OR 24,25,26-trihydroxycholecalciferol AND/OR 6,19-epidioxy-1alpha-hydroxy-6,19-dihydrocholecalciferol AND/OR 6,19-epidioxy-25-hydroxy-6,19-dihydrocholecalciferol AND/OR 24-Hydroxycalcitriol AND/OR 3 beta,7 alpha-Dihydroxy-5-cholestenoate AND/OR 3beta,7alpha-dihydroxycholest-5-en-27-oic acid AND/OR 5beta-spirostan-1beta,3alpha-diol AND/OR 7alpha,12alpha,24-trihydroxycholest-4-en-3-one AND/OR 7alpha,12alpha,26-trihydroxycholest-4-en-3-one;
10beta-Hydroxy-6beta-isobutyrylfuranoeremophilane AND/OR 5′-carboxy-alpha-chromanol AND/OR QH2 AND/OR Testolate;
1alpha,19,25-trihydroxy-10,19-dihydrocholecalciferol AND/OR 1alpha,25-dihydroxy-2alpha-hydroxymethyl-19-nor-20-epicholecalciferol AND/OR 1alpha,25-dihydroxy-2alpha-hydroxymethyl-19-norcholecalciferolcholecalciferol AND/OR 1alpha,25-dihydroxy-2beta-hydroxymethyl-19-nor-20-epicholecalciferol AND/OR 1alpha,25-dihydroxy-2beta-hydroxymethyl-19-norcholecalciferol AND/OR 3a,7a-Dihydroxycoprostanic acid AND/OR 3a,7a,12a-Trihydroxy-5b-cholestan-26-al AND/OR 3alpha,7alpha-dihydroxy-5beta-cholestan-26-oic acid AND/OR 3alpha,7alpha,12alpha-trihydroxy-5beta-cholestan-26-al AND/OR 3alpha,7alpha,24-trihydroxy-5beta-cholestan-26-al AND/OR 7alpha,12alpha,24-trihydroxy-5beta-cholestan-3-one AND/OR 7alpha,25,27-trihydroxycholesterol;
comparing the level of said biomarker to a control level; and
correlating the measurement with laminitis disease status, wherein a statistically relevant elevated level of said biomarker is present in said sample relative to said control level.

2. The method of claim 1, wherein said at least 1 biomarker is C17 Sphinganine.

3. The method for qualifying equine laminitis disease status of claim 2, wherein the method comprises identifying a further, different metabolic biomarker in a faecal sample of the subject and a combination of 2 different biomarkers qualifies equine laminitis disease status.

4. The method of any preceding claim, wherein the at least one biomarker is measured by LCMS.

5. A method for determining the course of progression of laminitis disease in an equine subject, comprising measuring at least 1 biomarker in a faecal sample from the subject, wherein said chemical biomarker is selected from:
C17 Sphinganine;
4-Hydroxyestrone sulfate AND/OR 1-O-alpha-D-Glucopyranosyl-D-mannitol AND/OR Melibiitol AND/OR (1(10)E,6a,9b)-9-(2-Methylpropanoyloxy)-1(10),4,11(13)-germacratrien-12,6-olide AND/OR Ubiquinone-2;
Citronellyl beta-sophoroside AND/OR 1alpha,11alpha,25-trihydroxycholecalciferol AND/OR 1alpha,11beta,25-trihydroxycholecalciferol AND/OR 1alpha,18,25-trihydroxycholecalciferol AND/OR 1alpha,2,25-trihydroxycholecalciferol AND/OR 1alpha,20,25-trihydroxycholecalciferol AND/OR 1alpha,22,25-trihydroxy-20-epicholecalciferol AND/OR 1alpha,23,25-trihydroxycholecalciferol AND/OR 1alpha,24,25-trihydroxycholecalciferol AND/OR 1alpha,25-dihydroxy-10,19-methano-23-oxacholecalciferol AND/OR 1alpha,25-dihydroxy-24a-homo-22-oxa-20-epicholecalciferol AND/OR 1alpha,25-dihydroxy-24a-homo-22-oxacholecalciferol AND/OR 1alpha,25,26-trihydroxycholecalciferol AND/OR 1alpha,26-trihydroxycholecalciferol AND/OR 22,24,25-trihydroxycholecalciferol AND/OR 23,24,25-trihydroxycholecalciferol AND/OR 23,25,26-trihydroxycholecalciferol AND/OR 24,25,26-trihydroxycholecalciferol AND/OR 6,19-epidioxy-1alpha-hydroxy-6,19-dihydrocholecalciferol AND/OR 6,19-epidioxy-25-hydroxy-6,19-dihydrocholecalciferol AND/OR 24-Hydroxycalcitriol AND/OR 3 beta,7 alpha-Dihydroxy-5-cholestenoate AND/OR 3beta,7alpha-dihydroxycholest-5-en-27-oic acid AND/OR 5beta-spirostan-1beta,3alpha-diol AND/OR 7alpha,12alpha,24-trihydroxycholest-4-en-3-one AND/OR 7alpha,12alpha,26-trihydroxycholest-4-en-3-one;
10beta-Hydroxy-6beta-isobutyrylfuranoeremophilane AND/OR 5′-carboxy-alpha-chromanol AND/OR QH2 AND/OR Testolate;
1alpha,19,25-trihydroxy-10,19-dihydrocholecalciferol AND/OR 1alpha,25-dihydroxy-2alpha-hydroxymethyl-19-nor-20-epicholecalciferol AND/OR 1alpha,25-dihydroxy-2alpha-hydroxymethyl-19-norcholecalciferolcholecalciferol AND/OR 1alpha,25-dihydroxy-2beta-hydroxymethyl-19-nor-20-epicholecalciferol AND/OR 1alpha,25-dihydroxy-2beta-hydroxymethyl-19-norcholecalciferol AND/OR 3a,7a-Dihydroxycoprostanic acid AND/OR 3a,7a,12a-Trihydroxy-5b-cholestan-26-al AND/OR 3alpha,7alpha-dihydroxy-5beta-cholestan-26-oic acid AND/OR 3alpha,7alpha,12alpha-trihydroxy-5beta-cholestan-26-al AND/OR 3alpha,7alpha,24-trihydroxy-5beta-cholestan-26-al AND/OR 7alpha,12alpha,24-trihydroxy-5beta-cholestan-3-one AND/OR 7alpha,25,27-trihydroxycholesterol
in the faecal sample from said subject at a first time point; measuring said same biomarker in a faecal sample from said patient at a second later time point; and comparing said measurement at said second time point to said measurement at said first time point, wherein a statistically relevant change in the concentration of the biomarker indicates either a progression or regression of laminitis in said subject.

6. The method of any previous claim, wherein the correlating step is performed by a software classification algorithm.

7. A competitive immunoassay for use in the detection of fumonisin in a pasture or food sample or a stool sample.

8. A method for determining the presence of mycotoxin fumonisin in a pasture or food sample, or an equine stool sample comprising:
undertaking a competitive immunoassay comprising immobilised fumonisin and an anti-fumonisin antibody conjugated to HRP wherein competition occurs between immobilised fumonisin and fumonisin in the sample for limited antibody binding sites and the captured antibody is detected: and
measuring a signal output, wherein a statistically relevant reduction in the signal output of the sample signifies the presence of fumonisin in the sample.

9. A fumonisin B1 antibody, antibody fragment and/or antibody conjugate for use in the identification of fumonisin in an equine food source or stool sample.

10. A fumonisin B1 monoclonal antibody for use in the diagnosis of laminitis in an equine.

11. An immuno-based assay comprising fumonisin B1 antibodies or fumonisin B1 anti-antibodies for use in monitoring the progression or regression of laminitis disease in an equine.

12. A composition for use in the treatment of laminitis comprising an agent which acts to reduce the bioavailability and/or action of mycotoxin fumonisin B1 in an equine.

13. The composition of claim 12, wherein the agent is selected from a binder, enzyme and/or absorbent, fumonisin B1 antibody or neutraliser.

14. The composition according to claim 12 or 13, wherein the composition further comprises enzyme-rich malt extract (ERME).
